(19) United States
(12) Patent Application Publication
(10) Pub. No.: US 2026/0248819 A1
(43) Pub. Date: Aug. 27, 2026

(54) MULTI-COMPONENT PHARMACEUTICAL COMPOSITIONS AND KITS CONTAINING NITRIC OXIDE RELEASING COMPOUNDS AND METHODS OF USING SAME

(71) Applicant: KNOW BIO, LLC, Morrisville, NC (US)

(72) Inventors: Rebecca Anthouard McDonald, Morrisville, NC (US); Nathan Allan Stasko, Morrisville, NC (US); Madyson Chambers, Durham, NC (US); Julie Katherine Geyer, Morrisville, NC (US)

(21) Appl. No.: 18/872,576

(22) PCT Filed: Jun. 7, 2023
(86) PCT No.: PCT/US2023/024729

§ 371 Date: Dec. 6, 2024

Related U.S. Application Data
(60) Provisional application No. 63/350,132, filed on Jun. 8, 2022.

(51) Int. Cl. A61K 31/655; A61K 31/135; A61K 31/397; A61K 31/407; A61K 31/41; A61K 31/47; A61K 31/546; A61K 31/7036; A61K 31/7048; A61K 38/12; A61P 31/04; A61P 31/10

(52) U.S. Cl. CPC A61K 31/655; A61K 31/135; A61K 31/397; A61K 31/407; A61K 31/41; A61K 31/47; A61K 31/546; A61K 31/7036; A61K 31/7048; A61K 38/12; A61P 31/04; A61P 31/10

(57) ABSTRACT

Provided herein are multi-component pharmaceutical compositions and kits comprising a first component comprising a nitric oxide (NO) releasing compound and a second component comprising a second therapeutic agent, wherein the second therapeutic agent comprises an antibiotic, an antifungal agent, or a combination thereof. In these compositions, a ratio of the first component to the second component provides an in vitro fractional inhibitory concentration index (FICI) of a combination of the first component and the second component of 1.0 or lower. The first component and second component can be formulated into a single, combined composition or can be maintained as separate compositions. Also described herein are methods of treating microbial infections and methods of preventing, reducing, or eliminating biofilm formation caused by bacteria.

### CROSS REFERENCE TO PRIORITY APPLICATION

This application claims priority to U.S. Provisional Application No. 63/350,132, filed Jun. 8, 2022, which is incorporated herein by reference in its entirety.

### FIELD

The present disclosure relates to multi-component pharmaceutical compositions and kits including two or more components, such as a nitric oxide releasing compound and an antimicrobial agent (e.g., an antibiotic or an antifungal agent). The present disclosure also relates to methods of using the contents of the multi-component pharmaceutical compositions and kits in treating microbial infections and inhibiting bacterial biofilm formation.

### BACKGROUND

Patients suffering from certain infections (e.g., chronic lung infections) are often prescribed multiple antibiotics simultaneously. Combinations of antibiotics are commonly described as antagonistic (compounds inhibit each other), indifferent (one compound alone is just as effective as the combination), additive (both compounds work equally well alone and in combination, so the combination is better than a single compound), or synergistic (compounds work better together than alone, so the effect is greater than the sum of its parts). Antagonistic combinations can have harmful effects on patients, accounting for patient morbidity in some instances. Therefore, it is important to evaluate whether antibiotics alter the efficacy of each other.

### SUMMARY

Provided herein are multi-component pharmaceutical compositions and kits, comprising at least two components that can work in a non-antagonistic manner to achieve the desired therapeutic effect (e.g., antimicrobial effect). The multi-component pharmaceutical compositions and kits can include a first component comprising a nitric oxide (NO) releasing compound and a second component comprising a second therapeutic agent, wherein the second therapeutic agent comprises an antibiotic, an antifungal agent, or a combination thereof. In these pharmaceutical compositions and kits, a ratio of the first component to the second component provides an in vitro fractional inhibitory concentration index (FICI) of a combination of the first component and the second component of 1.0 or lower (e.g., 0.5 or lower or 0.3 or lower). Details for calculating a FICI of the composition are provided in the Examples section below.

In some cases, a concentration of the second therapeutic agent in the second component is lower than the concentration of the second therapeutic agent needed alone (i.e., in the absence of the NO releasing compound) to exhibit an antimicrobial effect against a microbe. In some cases, the concentration of the second therapeutic agent in the second component is at least 10% lower, at least 20% lower, or at least 30% lower than the concentration of the second therapeutic agent needed alone to exhibit an antimicrobial effect against a microbe. In some cases, the NO releasing compound in the first component is present in an effective amount to sensitize or re-sensitize a microbe to the second therapeutic agent in the second component. Optionally, the first component and the second component are present in the pharmaceutical composition or kit as a single, combined composition. Optionally, the first component and the second component are present in the pharmaceutical composition or kit as separate compositions. In some cases, the first component is adapted for nebulization and the second component is adapted for intravenous administration.

The NO-releasing compound in the first component may comprise at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation. Optionally, the compound has the following structure:

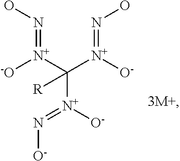

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)ₑH, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)ₑH, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M₊ is a pharmaceutically-acceptable cation, wherein a ratio of the compound to the cation is such that the overall net charge of the compound is neutral.

In some examples, the cation is selected from the group consisting of sodium, potassium, lithium, calcium, magnesium, ammonium, and substituted ammonium. Optionally, the compound has the following structure:

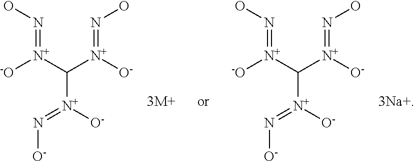

Optionally, the second therapeutic agent in the second component of the multi-component pharmaceutical composition or kit comprises an antibiotic. In some cases, the antibiotic can be selected from the group consisting of an aminoglycoside, a monobactam, a cephalosporin, a quinolone, a macrolide, a polymyxin, and a carbapenem. Optionally, the second therapeutic agent in the second component of the multi-component pharmaceutical composition or kit comprises an antifungal agent. The antifungal agent can be selected from the group consisting of a polyene, an azole, an allylamine, and an echinocandin.

Optionally, a molar equivalents concentration ratio of the NO releasing compound to the second therapeutic agent is from 0.1:1 to 10:1 (e.g., from 0.5:1 to 2:1). The multi-component pharmaceutical composition or kit and the individual components of the same as described herein can further comprise one or more additives (such as, for example, one or more preservatives, salts, chelators, viscosity modifiers, stabilizers, surfactants, antioxidants, buffering agents, or cosolvents).

Also described herein is a method of treating a microbial infection in a subject, comprising administering to the subject components of a multi-component pharmaceutical composition or kit as described herein. The method can comprise administering a first component comprising a nitric oxide (NO) releasing compound as described herein and administering to the subject a second component comprising a second therapeutic agent, wherein the second therapeutic agent comprises an antibiotic, an antifungal agent, or a combination thereof. In these methods, a ratio of the first component to the second component provides an in vitro fractional inhibitory concentration index (FICI) of a combination of the first component and the second component of 1.0 or lower (e.g., 0.5 or lower or 0.3 or lower).

Optionally, the first component including the NO releasing compound and the second component including the second therapeutic agent are each independently administered to the subject orally, parenterally, intravenously, via inhalation, intraperitoneally, intracranially, intraspinally, intrathecally, intraventricularly, intramuscularly, subcutaneously, sublingually, buccally, intracavitarly or transdermally. In some cases, the NO releasing compound and the second therapeutic agent are administered using the same mode of administration. In other cases, the NO releasing compound and the second therapeutic agent are administered using different modes of administration (e.g., the first component can be administered via a nebulizer and the second component can be administered intravenously). Optionally, the NO releasing compound and the second therapeutic agent are administered simultaneously. In some cases, the first component comprising the NO releasing compound and the second component comprising the second therapeutic agent are present in a single combined composition, and the single combined composition is administered to the subject. In other cases, the first component comprising the NO releasing compound and the second component comprising the second therapeutic agent are maintained as separate compositions, and are administered to the subject as separate compositions, either simultaneously (using the same or different modes of administration) or sequentially (using the same or different modes of administration). When administered sequentially, in some cases, the first component comprising the NO releasing compound is administered prior to administering the second component comprising the second therapeutic agent.

The microbial infection can be a bacterial infection. The bacterial infection can be caused by Gram-positive bacteria, Gram-negative bacteria, or atypical bacteria. Optionally, the bacterial infection is caused by Gram-positive bacteria species selected from the group consisting of Actinomyces species; Bacillus species; Clostridium species; Corynebacterium species; Enterococcus species; Leuconostoc species; Micrococcus species; Nocardia species; Propionibacterium species; Staphylococcus species; and Streptococcus species.

In some cases, the bacterial infection is caused by Gram-negative bacteria species selected from the group consisting of Acinetobacter species; Aeromonas species; Alcaligenes/Achromobacter species; Bacteroides species; Bartonella species; Bordetella species; Borrelia species; Brevundimonas species; Brucella species; Burkholderia species; Campylobacter species; Citrobacter species; Coxiella species; Ehrlichia species; Enterobacter species; Escherichia species; Francisella species; Haemophilus species; Helicobacter species; Klebsiella species; Leclercia species; Legionella species; Leptospira species; Listeria species; Moraxella species; Morganella species; Neisseria species; Orientia species; Pantoea species; Paracoccus species; Prevotella species; Proteus species; Providencia species; Pseudomonas species; Ralstonia species; Rickettsia species; Roseomonas species; Salmonella species; Serratia species; Shigella species; Sphingomonas species; Stenotrophomonas species; Treponema species; Ureaplasma species; Vibrio species; and Yersinia species. Optionally, the Gram-negative bacteria species comprises Pseudomonas aeruginosa.

In some cases, the bacterial infection is caused by atypical bacteria species selected from the group consisting of Mycobacteria species; Chlamydia/Chlamidophila species; and Mycoplasma species. Optionally, the bacterial infection is caused by antibiotic-resistant bacteria.

Optionally, the method is performed under aerobic conditions, anaerobic conditions, or microaerobic conditions. In the method, a concentration of the second therapeutic agent in the second component administered to the subject can be lower than the concentration of the second therapeutic agent needed alone to exhibit an antimicrobial effect against a microbe in the subject. In some cases, the concentration of the second therapeutic agent in the second component administered to the subject is at least 10% lower, at least 20% lower, or at least 30% lower than the concentration of the second therapeutic agent needed alone to exhibit an antimicrobial effect against a microbe in the subject. In some cases, the NO releasing compound in the first component sensitizes or re-sensitizes a microbe to the second therapeutic agent.

Further described herein is a method of preventing, reducing, or eliminating biofilm formation caused by bacteria, comprising contacting bacteria with contents of a multi-component pharmaceutical composition or kit as described herein. The method can comprise administering a first component comprising a nitric oxide (NO) releasing compound as described herein and a second component comprising a second therapeutic agent, wherein the second therapeutic agent comprises an antibiotic, an antifungal agent, or a combination thereof. In this method, a ratio of the first component to the second component provides an in vitro fractional inhibitory concentration index (FICI) of a combination of the first component and the second component of 1.0 or lower. Additionally described herein is a method of treating a surface to prevent, reduce, or eliminate biofilm formation caused by bacteria, comprising contacting a surface with a contents of a multi-component pharmaceutical composition or kit as described herein. The method can comprise administering a first component comprising a nitric oxide (NO) releasing compound as described herein and a second component comprising a second therapeutic agent, wherein the second therapeutic agent comprises an antibiotic, an antifungal agent, or a combination thereof. In this method, a ratio of the first component to the second component provides an in vitro fractional inhibitory concentration index (FICI) of a combination of the first component and the second component of 1.0 or lower.

The details of one or more embodiments are forth in the drawings and the description below. Other features, objects, and advantages will be apparent from the description and drawings, and from the claims.

### DESCRIPTION OF THE DRAWINGS

FIGS. 1A and 1B are graphs showing the efficacy of MD3 combined with a second antibiotic against P. aeruginosa strain PAK grown under aerobic (FIG. 1A) or anaerobic (FIG. 1B) conditions. Abbreviations: ABX, antibiotic; TOB, tobramycin; AZT, aztreonam; CIP, ciprofloxacin; CST, colistin sulfate; CAZ, ceftazidime; MER, meropenem; GaCi, gallium citrate; GEN, gentamicin.

FIG. 2 is a graph showing the efficacy of MD3 combined with a second antibiotic against NTM species grown under aerobic conditions. Abbreviations: ABX, antibiotic; AMK, amikacin; CLR, clarithromycin; GaCi, gallium citrate. NT, not tested.

FIG. 3 is a graph showing the efficacy of MD3 combined with a second antibiotic against S. aureus grown under aerobic conditions. Abbreviations: TOB, tobramycin; GaCi, gallium citrate.

FIG. 4 is a graph showing the number of remaining P. aeruginosa bacteria per mL of sample after treatment with varying concentrations of MD3. Biofilm-associated bacteria measurements are shown in the left group of bars (bars 1-6, starting from the left) and planktonic bacteria measurements are shown in the right group of bars (bars 7-12, starting from the left). The starting colony forming units per mL (CFU/mL), prior to treatment with MD3, was 9.50E+05 and is indicated by the dotted line.

FIG. 5 is a graph showing that MD3 eradicates P. aeruginosa biofilms. Aerobic and anaerobic biofilms of P. aeruginosa were treated with MD3 (left group of bars) or tobramycin (right group of bars) to determine the minimum biofilm eradication concentration (MBEC), which is defined as a 3-log reduction in biofilm-associated CFUs. In each group of bars, the left bar represents aerobic conditions and the right bar represents anaerobic conditions. The limit of detection is indicated by the dotted line.

### DETAILED DESCRIPTION

Described herein are multi-component pharmaceutical compositions and multi-component kits including two or more components. A first component in the pharmaceutical composition or multi-component kit includes a nitric oxide (NO) releasing compound. A second component in the pharmaceutical composition multi-component kit includes an antimicrobial agent, such as an antibiotic or an antifungal agent. Also described herein are methods of using the pharmaceutical composition and/or the components of the multi-component kits in treating microbial infections and inhibiting bacterial biofilm formation.

Notably, the pharmaceutical compositions and multi-component kits can be designed and tailored, with respect to modes of administration, such that the maximum therapeutic effect can be attained. By way of example and as further described herein, the compositions and multi-component kits composed of two or more components can be prepared such that the components are combined into a single composition prior to administration or are kept separate and administered as separate compositions to the subject. When combined, the combined composition is administered via a single effective mode of administration as further described herein. When the components are separate (i.e., when the components are administered as separate compositions), the compositions can be administered simultaneously by the same mode of administration or different modes of administration, or can be administered sequentially using the same mode of administration or different modes of administration. Such administration methods are further described herein. The flexibility in mode of administration adds to the desirability of the pharmaceutical compositions and kits described herein in treating and preventing microbial infections and inhibiting bacterial biofilm formation, as the pharmaceutical composition and kit components are capable of imparting multi-faceted impact to a subject in a single treatment regimen. The pharmaceutical composition and kit components can be adapted for the desired type of delivery. In some cases, the first component is adapted for nebulization and the second component is adapted for intravenous administration.

The pharmaceutical compositions and kits described herein can exhibit enhanced, unexpected antimicrobial effects due to the impact of the NO releasing compound in the first component of the pharmaceutical composition and/or kit. First, the NO releasing compound, on its own, is a contributor of an active pharmaceutical ingredient to the composition, due to its release of nitric oxide. The NO releasing compound works collaboratively with a second therapeutic agent, such as an antimicrobial agent, to enhance the therapeutic impact of the treatment. In some cases, the NO releasing compound and the second therapeutic agent can work additively, such that the delivered combination of the two agents is more effective than the delivery of one agent. In some cases, the NO releasing compound and the second therapeutic agent can work synergistically, such that the delivered combination of the two agents has an effect that is greater than the sum of its parts. In other cases, the NO releasing compound and the second therapeutic agent can work indifferently, such that one compound alone is just as effective as the combination.

In addition to this collaborative impact of the agents (be it additive, synergistic, or even indifferent in some cases), the NO releasing compound can cause additional effects that allow the second therapeutic agent to enhance its impact on a microbe. For example, the NO released from the NO releasing compound can damage the resistance mechanisms of the bacteria, thereby increasing antimicrobial susceptibility and decreasing resistance over time. Such a combined impact, i.e., increasing antimicrobial susceptibility and imparting antimicrobial effects, results in a greater than additive result on treating the bacterial infection (in other words a synergistic effect). Additionally, the NO released from the NO releasing compound can restore the breakpoint susceptibility of the microbe, such that an organism that is multidrug resistant and not susceptible to an antibiotic can be made effective. Such a combined impact is a synergistic effect.

The released NO also has direct effects on antimicrobial biofilms. Biofilm formation causes significant complications across a broad spectrum of issues, including, but not limited to, in the treatment of bacterial infections. For example, Pseudomonas aeruginosa and non-tuberculosis mycobacteria (among a host of other pathogens) possess the ability to establish biofilms in a variety of locations, including within the lungs of CF patients and on surfaces of medical devices. Such biofilms, once formed, are difficult to control and disrupt and represent one of the leading causes of hospital-acquired infections. Many antibiotics are ineffective in treating infections stemming from bacteria growing in biofilms or require increased amounts of drug to treat biofilm-associated bacteria as compared to planktonic bacteria. Surprisingly, the NO from the NO releasing compound in the composition enables the co-administered (be it simultaneously or sequentially) antibiotic to be effective against the bacteria. In some instances, the NO loosens the biofilm matrix, impacts the redox state of the biofilm, and activates quiescent cells and thus promoting an active metabolism.

Notably, the use of some amount of NO allows a decreased amount of the second therapeutic agent to be administered, and still have the same effect as the second therapeutic agent (e.g., antimicrobial agent) administered alone. Due to NO's effectiveness against the microbe's machinery, the amount of antimicrobial agent needed to produce an antimicrobial effect is at least 10% less, at least 15% less, at least 20% less, at least 25% less, at least 30% less, at least 35% less, or at least 40% less than the amount of an antimicrobial agent administered alone, while still having the same or greater impact. Such multi-component kits displaying these enhanced antimicrobial effects are further described herein.

### I. Multi-Component Pharmaceutical Compositions and Kits

A multi-component pharmaceutical composition or a multi-component kit as described herein includes at least two compositions that work in a non-antagonistic manner to achieve the desired therapeutic effect (e.g., antimicrobial effect). In some examples, the components within the multi-component pharmaceutical composition or kit work in a synergistic manner with respect to imparting antimicrobial effects. In some examples, the components within the multi-component pharmaceutical composition or kit work in an additive manner with respect to imparting antimicrobial effects. In other examples, the components within the multi-component pharmaceutical composition or kit work in an indifferent manner with respect to imparting antimicrobial effects.

The multi-component pharmaceutical composition or kit as described herein includes a first component, which includes a nitric oxide (NO) releasing compound as further detailed below. The first component can exhibit antimicrobial characteristics, including antiviral, antibacterial, and antifungal characteristics. The firm component described herein can further possess anti-inflammatory properties and other beneficial therapeutic properties. The multi-component pharmaceutical composition or kit as described herein further includes a second component including a second therapeutic agent. The second therapeutic agent can be an antimicrobial agent, such as an antibiotic, an antifungal agent, or a combination thereof. The components of the pharmaceutical composition and kit are further described below.

The multi-component pharmaceutical composition or kit components can be packaged and administered in a variety of manners and modes, respectively. As such, the multi-component pharmaceutical composition or kit components described herein can be tailored to achieve the maximum antimicrobial impact to benefit the subject. By way of example, the first component and the second component of the multi-component pharmaceutical composition or kit can be prepared such that the two compositions are within a single, combined composition. That single, combined composition can, in turn, be administered to a subject via the desired mode of administration (e.g., orally, parenterally, intravenously, via inhalation, intraperitoneally, intracranially, intraspinally, intrathecally, intraventricularly, intramuscularly, subcutaneously, sublingually, buccally, intracavitarly or transdermally).

In other examples, the first component and the second component of the multi-component pharmaceutical composition or kit can be prepared such that the two compositions are maintained as separate components (i.e., not otherwise mixed prior to administering to the subject). In some instances, the first component and the second component are housed in separate containers and may be subjected to different storage conditions prior to administration, as required by the individual composition components and as determined by one of ordinary skill in the art based on the disclosure provided herein. Optionally, the first component and the second component, separately, can be administered to a subject simultaneously. Such administration can be performed via the same mode of administration or via different modes of administration. For example, the first component can be administered to the subject via inhalation while the second component is administered to the subject orally. In other examples, the first component can be administered via a nebulizer and the second component is administered intravenously. Optionally, the first component and the second component can be administered to a subject sequentially. Such sequential administration can be performed via the same mode of administration (i.e., the first and second components are administered sequentially but using the same mode of administration) or via different modes of administration (i.e., the first and second components are administered sequentially and using different modes of administration). In some examples, the first component is administered prior to the second component. In other examples, the second component is administered prior to the first component.

a. Nitric Oxide Donor Component

An active pharmaceutical ingredient delivered via the first component described herein is nitric oxide and can be included in the first component in the form of a compound that releases nitric oxide (NO) (e.g., nitric oxide donors, nitric oxide releasing prodrugs, or compounds necessary to facilitate the endogenous generation of nitric oxide). As described herein, the first component in the form of a compound that releases NO can exhibit antimicrobial characteristics, including antiviral, antibacterial, and antifungal characteristics. The firm component described herein can further possess anti-inflammatory properties and other beneficial therapeutic properties.

The NO release can be initiated thermally or via any of the degradation strategies for the labile portion of N-diazeniumdiolates, nitrosamines, hydroxyl nitrosamines, nitrosothiols, hydroxylamines, hydroxyureas, metal complexes, organic nitrites and organic nitrates. See, Wang, P. G., et al., Nitric Oxide Donors. For Pharmaceutical and Biological Applications; Wliey-VCH. Weinheim, Germany, 2005; and Wang. P. G., et al., Chem. Rev., 102, 1091-1134 (2002). In some embodiments, the NO donor is a N-diazeniumdiolate (i.e., a 1-amino-substituted deazen-1-ium-1,2-diolate). N-Diazeniumdiolates are particularly attractive as NO donors due to their ability to generate NO spontaneously under biological conditions. See Hrabie, J. A. and Keefer, L. K. Chem. Rev., 102, 1135-1154 (2002); and Napoli, C. and Ianarro, L. J., Annu. Rev. Pharmacol. Toxicol., 43, 97-123 (2003). Several N-diazeniumdiolate compounds have been synthesized using a range of nucleophilic residues that encompass primary and secondary amines, polyamines, and secondary amino acids, See Hrabie, J. A., and Keefer L. K. Chem. Rev., 102, 1135-1154 (2002). In the formation of the N-diazeniumdiolate, one equivalent of amine reacts with two equivalents of nitric oxide under elevated pressure. A base (e.g., an alkoxide like methoxide) removes a proton from the amine nitrogen to create the anionic, stabilized N(O)NO group. While stable under ambient conditions, N-diazeniumdiolates decompose spontaneously in aqueous media to generate NO at rates dependent upon pH, temperature, and/or the structure of the amine moiety.

In some cases, the first component can include at least one nitric oxide releasing compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

As stated above, the compounds described herein can include at least one nitric oxide releasing functional group. Although various NO donors (e.g., diazeniumdiolates, S-nitrosothiols, metal nitrosyls, organic nitrates) are known to provide for controlled exogenous NO release, the diazeniumdiolate functional group (NONOate) in the compounds disclosed herein are attractive because of their good stability and facile storage, and because they spontaneously undergo proton-triggered dissociation under physiological conditions to regenerate nitric oxide. Certain compounds include two diazeniumdiolate groups on one carbon atom, each having a charge and each with an associated pharmaceutically-acceptable cation to balance the charge on the diazeniumdiolate groups. The compounds are small molecules (having a molecular weight of 500 g/mol or less, without the cation, as further described below) that release nitric oxide (NO) and exhibit antimicrobial characteristics, including antiviral, antibacterial, and antifungal characteristics, anti-inflammatory properties, and other beneficial therapeutic properties.

Optionally, the compound has the following structure, as represented by Formula I.

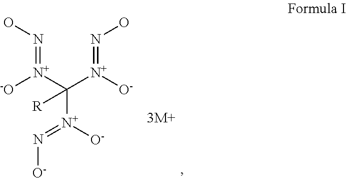

In Formula I, R is hydrogen, deuterium, C₁₋₁₂ alkyl, aryl, heteroaryl, alkylaryl, arylalkyl, or carbonyl. Optionally R is substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)ₑH, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H, wherein a, b, c, d, e, and f are each independently selected from an integer of 0, 1, 2, 3, 4, 5, 6, 7, 8, 9, or 10.

Additionally in Formula I, M₊ is a cation. For example, M₊ can be a pharmaceutically acceptable cation. Optionally, the cation is selected from the group consisting of sodium, potassium, lithium, calcium, magnesium, and quaternary ammonium salts (e.g., ammonium or substituted ammonium).

In these compositions, a ratio of the compound to the cation is such that the overall net charge of the compound is neutral. In cases where M₊ is a cation with a valence other than one, a ratio of the compound to the cation is such that the total positive charge equals the total negative charge. By way of example, for a compound having a total charge of negative three, and a cation with a total charge of positive one, there would be one compound and three cations.

For example, the compound can be represented by Structure I-A, as shown below:

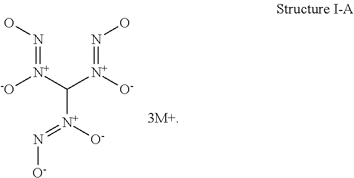

As shown above in Structure I-A, the compound has a total charge of negative three. Therefore, three cations (i.e., 3 M₊) are present to balance the charge of the compound (i.e., the total positive charge equals the total negative charge).

An example of Structure I-A includes the following compound:

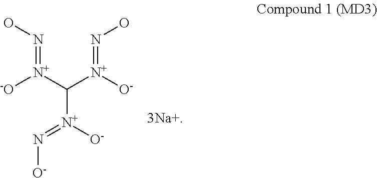

The compound can have a molecular weight below 500 g/mol, not including the associated cation (e.g., the associated pharmaceutically-acceptable cation). For example, the compound can have a molecular weight of 450 g/mol or less, 400 g/mol or less, 350 g/mol or less, 300 g/mol or less, 250 g/mol or less, or 200 g/mol or less. Optionally, the molecular weight of the compound, excluding the associated cation, can be from 100 g/mol to below 500 g/mol, from 120 g/mol to 450 g/mol, from 150 g/mol to 400 g/mol, or from 175 g/mol to 350 g/mol.

Additional details regarding the mechanism of action of the compounds described herein, including their nitric oxide delivery properties, and advantageous properties of the compounds (e.g., storage stability) are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bonded through a single, terminal ether linkage. The term aryloxy as used herein is an aryl group bonded through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bonded through a single, terminal ether linkage.

The term hydroxy as used herein is represented by the formula OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

The C-diazeniumdiolates described herein are pH-triggered NO-releasing donors (also referred to herein as NO-releasing compounds or NO-releasing agents). Reacting with protons under physiological conditions (e.g., 37° C., pH 7.4), 1 mole of Compound 1 (MD3) generates two moles of NO and 2 to 3 moles of nitroxyl compounds.

In several embodiments, the NO-releasing compounds are stable at a variety of temperatures from frozen to room temperature 25° C. (e.g., −20° C., 0° C., 5° C., 20° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year, 2 years, or greater)).

In some cases, the compound has a total releasable NO storage in a range of 0.1 μmol to 23.0 μmol of NO per mg of the compound (e.g., from 0.1 μmol to 15 μmol per mg of the compound, from 0.5 μmol to 7.5 μmol per mg of the compound, from 1 μmol to 7.0 μmol per mg of the compound, from 1.5 μmol to 6.5 μmol per mg of the compound, from 2.0 μmol to 6.0 μmol per mg of the compound, from 2.5 μmol to 5.5 μmol per mg of the compound, or from 3.0 μmol to 5.0 μmol per mg of the compound). For example, the total releasable NO storage of the compounds for use in the composition can be 0.1 μmol, 0.2 μmol, 0.3 μmol, 0.4 μmol, 0.5 μmol, 0.6 μmol, 0.7 μmol, 0.8 μmol, 0.9 μmol, 1.0 μmol, 1.1 μmol, 1.2 μmol, 1.3 μmol, 1.4 μmol, 1.5 μmol, 1.6 μmol, 1.7 μmol, 1.8 μmol, 1.9 μmol, 2.0 μmol, 2.1 μmol, 2.2 μmol, 2.3 μmol, 2.4 μmol, 2.5 μmol, 2.6 μmol, 2.7 μmol, 2.8 μmol, 2.9 μmol, 3.0 μmol, 3.1 μmol, 3.2 μmol, 3.3 μmol, 3.4 μmol, 3.5 μmol, 3.6 μmol, 3.7 μmol, 3.8 μmol, 3.9 μmol, 4.0 μmol, 4.1 μmol, 4.2 μmol, 4.3 μmol, 4.4 μmol, 4.5 μmol, 4.6 μmol, 4.7 μmol, 4.8 μmol, 4.9 μmol, 5.0 μmol, 5.1 μmol, 5.2 μmol, 5.3 μmol, 5.4 μmol, 5.5 μmol, 5.6 μmol, 5.7 μmol, 5.8 μmol, 5.9 μmol, 6.0 μmol, 6.1 μmol, 6.2 μmol, 6.3 μmol, 6.4 μmol, 6.5 μmol, 6.6 μmol, 6.7 μmol, 6.8 μmol, 6.9 μmol, 7.0 μmol, 7.1 μmol, 7.2 μmol, 7.3 μmol, 7.4 μmol, 7.5 μmol, 7.6 μmol, 7.7 μmol, 7.8 μmol, 7.9 μmol, 8.0 μmol, 8.1 μmol, 8.2 μmol, 8.3 μmol, 8.4 μmol, 8.5 μmol, 8.6 μmol, 8.7 μmol, 8.8 μmol, 8.9 μmol, 9.0 μmol, 9.1 μmol, 9.2 μmol, 9.3 μmol, 9.4 μmol, 9.5 μmol, 9.6 μmol, 9.7 μmol, 9.8 μmol, 9.9 μmol, 10.0 μmol, 10.1 μmol, 10.2 μmol, 10.3 μmol, 10.4 μmol, 10.5 μmol, 10.6 μmol, 10.7 μmol, 10.8 μmol, 10.9 μmol, 11.0 μmol, 11.1 μmol, 11.2 μmol, 11.3 μmol, 11.4 μmol, 11.5 μmol, 11.6 μmol, 11.7 μmol, 11.8 μmol, 11.9 μmol, 12.0 μmol, 12.1 μmol, 12.2 μmol, 12.3 μmol, 12.4 μmol, 12.5 μmol, 12.6 μmol, 12.7 μmol, 12.8 μmol, 12.9 μmol, 13.0 μmol, 13.1 μmol, 13.2 μmol, 13.3 μmol, 13.4 μmol, 13.5 μmol, 13.6 μmol, 13.7 μmol, 13.8 μmol, 13.9 μmol, 14.0 μmol, 14.1 μmol, 14.2 μmol, 14.3 μmol, 14.4 μmol, 14.5 μmol, 14.6 μmol, 14.7 μmol, 14.8 μmol, 14.9 μmol, 15.0 μmol, 15.1 μmol, 15.2 μmol, 15.3 μmol, 15.4 μmol, 15.5 μmol, 15.6 μmol, 15.7 μmol, 15.8 μmol, 15.9 μmol, 16.0 μmol, 16.1 μmol, 16.2 μmol, 16.3 μmol, 16.4 μmol, 16.5 μmol, 16.6 μmol, 16.7 μmol, 16.8 μmol, 16.9 μmol, 17.0 μmol, 17.1 μmol, 17.2 μmol, 17.3 μmol, 17.4 μmol, 17.5 μmol, 17.6 μmol, 17.7 μmol, 17.8 μmol, 17.9 μmol, 18.0 μmol, 18.1 μmol, 18.2 μmol, 18.3 μmol, 18.4 μmol, 18.5 μmol, 18.6 μmol, 18.7 μmol, 18.8 μmol, 18.9 μmol, 19.0 μmol, 19.1 μmol, 19.2 μmol, 19.3 μmol, 19.4 μmol, 19.5 μmol, 19.6 μmol, 19.7 μmol, 19.8 μmol, 19.9 μmol, 20.0 μmol, 20.1 μmol, 20.2 μmol, 20.3 μmol, 20.4 μmol, 20.5 μmol, 20.6 μmol, 20.7 μmol, 20.8 μmol, 20.9 μmol, 21.0 μmol, 21.1 μmol, 21.2 μmol, 21.3 μmol, 21.4 μmol, 21.5 μmol, 21.6 μmol, 21.7 μmol, 21.8 μmol, 21.9 μmol, 22.0 μmol, 22.1 μmol, 22.2 μmol, 22.3 μmol, 22.4 μmol, 22.5 μmol, 22.6 μmol, 22.7 μmol, 22.8 μmol, 22.9 μmol, or 23.0 μmol per mg of the compound.

The compound can have a total duration of NO release, upon activation, in a range of 0.1 to 60 hours. In some cases, the NO release may occur over a period of about 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, or 60 hours. In some embodiments, within 2 hours of being added to a phosphate buffered saline (PBS) buffer solution, the compounds release greater than or equal to about: 25%, 50%, 75%, 85%, 90%, 95%, 100%, or ranges including and/or spanning the aforementioned values, their total wt. % of bound NO. Optionally, the compound has a total NO release of 0.1-8.0 μmol of NO per mg of the compound after 4 hours of the initiation of NO release (also referred to as “activation”).

In some embodiments, the compounds have a release rate per hour using chemiluminescent based nitric oxide detection of less than or equal to about: 0.2%, 0.5%, 1.0%, 1.5%, 2.5%, 5.0%, 10%, or ranges including and/or spanning the aforementioned values.

Optionally, the compound for use in the compositions described herein has a NO release half-life in the range of 0.01-24 hours. In several embodiments, the NO release half-life is equal to or at least about: 0.01 hours, 0.1 hours, 0.25 hours, 0.5 hours, 1 hour, 2 hours, 3 hours, 4 hours, 5 hours or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours or ranges including and/or spanning the aforementioned values.

Optionally, the first component of the multi-component pharmaceutical composition can be or include a composition as described in PCT/US2023/019807, entitled “Buffering Agent-Containing Compositions and Methods of Using Same,” which is incorporated herein by reference in its entirety.

b. Second Therapeutic Agent Component

As described above, the second component described herein includes a second therapeutic agent. The second therapeutic agent can be, in some instances, an antibiotic. Suitable antibiotics can include any antibiotic effective for treating a bacterial infection and/or inhibiting or disrupting a biofilm and include, for example, tetracyclines (e.g., minocycline), quinolones (e.g., ciprofloxacin, levofloxacin, and nalidixic acid), aminoglycosides (e.g., amikacin, gentamycin, kanamycin, and tobramycin), carbapenems (e.g., meropenem), cephalosporins (e.g., ceftriaxone and ceftazidime), macrolides (e.g., erythromycin and clarithromycin), polypeptides (e.g., colistin and polymxin B), sulfonamides (e.g., sulfamethoxazole), glycylcyclines (e.g., tigecycline), beta lactams (e.g., penams), lipopeptides (e.g., daptomycin), oxazolidinones (e.g., linezolid), trimethoprim, and monobactams.

For example, the second component described herein can include an antibiotic, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; aztreonam; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodium; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In some cases, the second therapeutic agent can be an antifungal agent. Optionally, the antifungal agent can be selected from the group consisting of a polyene, an azole, an allylamine, and an echinocandin. Exemplary antifungal agents include, for example, amphoterican B, nystatin, flucytosin, natamycin, ketoconazole, econoazole, miconazole, itraconazole, fluconazole, clotrimazole, griseofulvin, oxiconazole, terconazole, tioconazole, clotrimazole, silver sulfadiazine, ciclopirox olamine, and terbinafine.

Optionally, the second component of the multi-component pharmaceutical composition can be or include a composition as described in PCT/US2023/019807, entitled “Buffering Agent-Containing Compositions and Methods of Using Same,” which is incorporated herein by reference in its entirety.

c. Optional Additives

The multi-component pharmaceutical compositions and kits and/or one or more individual components/compositions within the multi-component pharmaceutical compositions and kits described herein can further include one or more additives. The one or more additives can include, for example, one or more preservatives, salts, chelators, stabilizers, surfactants, antioxidants (e.g., N-acetylcysteine or glutathione), buffering agents, and/or cosolvents. The multi-component pharmaceutical compositions and kits and/or individual compositions within the kits, if desired, can also contain wetting or emulsifying agents, lubricants, glidants, emollients, humectants, thickeners, and/or flavoring agents.

In some cases, the one or more additives can include viscosity-reducing agents, natural and synthetic anti-biofilm agents (e.g., chitosan), biofilm dispersing agents, natural and synthetic anti-quorum-sensing agents (e.g., autoinducer-2 or N-acyl homo-serine lactones), siderophores, iron chelators, iron mimetics (e.g., gallium (Ga) and gallium-containing compounds, such as gallium azoles (Ga-azoles)), anti-persister cell agents (e.g., 4-(4,7-di-methyl-1,2,3,4-tetrahydro-naphthalene-1-yl) pentanoic acid (DMNP)), antimicrobial peptides (AMPs) (e.g., LL-37 or lactoferricin), efflux pump inhibitors, and/or bacteriophage therapy.

Optionally, the additives can be present in the multi-component pharmaceutical compositions or kits in an amount of less than 1 wt. %. For example, the amount of the additive can be less than 0.9 wt. %, less than 0.8 wt. %, less than 0.7 wt. %, less than 0.6 wt. %, less than 0.5 wt. %, less than 0.4 wt. %, less than 0.3 wt. %, less than 0.2 wt. %, or less than 0.1 wt. %. The amount of additive can optionally be 0.1 to 0.9 wt. %, 0.2 to 0.8 wt. %, or 0.3 to 0.7 wt. %.

Viscosity modifiers can optionally be included in the multi-component pharmaceutical compositions and kits and/or one or more individual compositions within the multi-component pharmaceutical composition and kits as described herein. Optionally, the viscosity modifiers can be included in the multi-component pharmaceutical compositions or kits in an amount of up to 5 wt. % (e.g., 0.1 wt. % to 5 wt. %, 0.5 wt. % to 4.5 wt. %, 1.0 wt. % to 4.0 wt. %, 1.5 wt. % to 3.5 wt. %, or 2.0 wt. % to 3.0 wt. %). For example, the viscosity modifier can be 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, or 4.5 wt. %, or 5.0 wt. %.

d. Synergistic, Additive, and Indifferent Compositions

As outlined above, in some embodiments, the multi-component pharmaceutical composition or kit components described herein can be synergistic, additive, or indifferent with respect to the relationship of the first component to the second component. Specifically, a ratio of the first component to the second component provides an in vitro fractional inhibitory concentration index (FICI) of a combination of the first component and the second component of 1.0 or lower (e.g., 0.5 or lower or 0.3 or lower). In some cases, FICI values of 0.5 or less are considered synergistic, values of 0.51 to 1 are considered additive, and values of greater than 1 to 4 are considered indifferent.

The identities and amounts of the first component and second component of each composition can be selected such that the desired relationship (be it synergistic, additive, or indifferent) is achieved. For example, a molar equivalents concentration ratio of the NO releasing compound to the second therapeutic agent can be selected such that the desired relationship is achieved. Optionally, a molar equivalents concentration ratio of the NO releasing compound to the second therapeutic agent is from 0.1:1 to 10:1 (e.g., from 0.5:1 to 2:1). In some cases, the molar equivalents concentration ratio of the NO releasing compound to the second therapeutic agent is 0.1:1, 0.5:1, 1:1, 2:1, 3:1, 4:1, 5:1, 6:1, 7:1, 8:1, 9:1, or 10:1.

A concentration of the second therapeutic agent in the second component can be lower than the concentration of the second therapeutic agent needed alone to exhibit an antimicrobial effect against a microbe. In some cases, the concentration of the second therapeutic agent in the second component is at least 10% lower than the concentration of the second therapeutic agent needed alone to exhibit an antimicrobial effect against a microbe.

In other cases, the concentration of the second therapeutic agent in the second component is at least 20% lower than the concentration of the second therapeutic agent needed alone to exhibit an antimicrobial effect against a microbe. In still other cases, the concentration of the second therapeutic agent in the second component is at least 30% lower than the concentration of the second therapeutic agent needed alone to exhibit an antimicrobial effect against a microbe. Optionally, the NO releasing compound is present in an effective amount to sensitize or re-sensitize a microbe to the second therapeutic agent.

### II. Methods of Making the Compounds

The compounds described herein can be prepared in a variety of ways. The compounds can be synthesized using, for example, various synthetic methods. At least some of these methods are known in the art of synthetic organic chemistry. The compounds described herein can be prepared from readily available starting materials. Optimum reaction conditions may vary with the particular reactants or solvents used, but such conditions can be determined by one skilled in the art.

Variations on Formula I and the compounds described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of ordinary skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H-NMR or ¹³C-NMR), infrared spectroscopy (IR), spectrophotometry (e.g., UV-visible), or mass spectrometry (MS), or by chromatography such as high performance liquid chromatography (HPLC) or thin layer chromatography (TLC).

Optionally, the compounds described herein can be prepared according to the methods of synthesis described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

### III. Pharmaceutical Compositions

In some cases, the multi-component compositions, including the multi-component kits, are pharmaceutical compositions. In cases of pharmaceutical compositions and in some cases where the multi-component kit compositions are pharmaceutical compositions, the compositions can include a buffering agent. Buffering agents can be included to control the pH of the composition. In some examples, the buffering agent is included to maintain the pH of the composition between 5.5 and 8.5. For example, the buffering agent can be included to maintain the pH of the composition between 6.0 to 8.0, 6.7 to 7.5, or 7.0 to 7.5 (e.g., 7.4).

The buffering agent can have a buffering strength of from 0.1 to 2.0 molar equivalents (e.g., from 0.1 to 1.5 molar equivalents, from 0.2 to 1.25 molar equivalents, or from 0.3 to 1.0 molar equivalents). For example, the buffering agent can have a buffering strength of 0.1 molar equivalents, 0.2 molar equivalents, 0.3 molar equivalents, 0.4 molar equivalents, 0.5 molar equivalents, 0.6 molar equivalents, 0.7 molar equivalents, 0.8 molar equivalents, 0.9 molar equivalents, 1.0 molar equivalent, 1.1 molar equivalents, 1.2 molar equivalents, 1.3 molar equivalents, 1.4 molar equivalents, 1.5 molar equivalents, 1.6 molar equivalents, 1.7 molar equivalents, 1.8 molar equivalents, 1.9 molar equivalents, or 2.0 molar equivalents.

In general, the buffering agent can be any buffering agent generally regarded as safe for use as inactive ingredients suitable for inhalation. In some embodiments, the buffering agent for use in the compositions described herein includes a phosphate buffering agent. Examples of suitable phosphate buffering agents include, for example, 0.01-1 M phosphate buffering agents. Optionally, the phosphate buffering agent is a potassium phosphate buffer. The counter cation of the buffering agent for use in the compositions can be selected to enhance the biologic activity of the composition or to minimize complexity in the analytical characterization of the composition.

Specifically, certain examples of the compounds described herein, such as MD3 (as further described below), includes sodium cations as counterions. When measuring the amount of the compound in a certain formulation, such as an aerosol formulation, the amount of the sodium cation is calculated. Under these circumstances, the presence of sodium in a buffering agent (e.g., a sodium phosphate buffering agent) would obfuscate the compound calculation. In other examples, the presence of potassium in a buffering agent may have an impact on proton pumps and the resulting pH of the epithelial lining fluid when administered to a human. Potassium does not increase the alkalinity of the composition, which is beneficial as an increase in alkalinity would interfere with NO release from a nitric oxide releasing compound.

One or more buffering agents can be included in the composition, including acetate buffers, benzoate buffers, citrate buffers, lactate buffers, maleate buffers, and tartrate buffers. Optionally, the one or more buffering agents includes a HEPES ((4-(2-hydroxyethyl)-1-piperazineethanesulfonic acid) buffering agent.

In some examples, the composition is substantially free from carbonate buffering agents. In some examples the composition is substantially free from hydrochloric acid, sulphuric acid, or citric acid. As used herein, the term “substantially free” from an indicated component (e.g., carbonate buffers, hydrochloric acid, sulphuric acid, and/or citric acid), means that the pharmaceutical composition can include less than 1%, less than 0.1%, less than 0.01%, less than 0.001%, or less than 0.0001% of the component (e.g., carbonate buffering agents, hydrochloric acid, sulphuric acid, and/or citric acid) based on the weight of the pharmaceutical composition.

In cases of pharmaceutical compositions and in some cases where the multi-component kit compositions are pharmaceutical compositions, the compositions can include a pharmaceutically acceptable carrier. As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended mode or modes of administration for the composition.

Suitable liquid carriers can be aqueous or non-aqueous carriers. Examples of suitable non-aqueous carriers include propylene glycol, polyethylene glycol, and oils, including those of petroleum, animal, vegetable or synthetic origin, such as peanut oil, soybean oil, mineral oil, sesame oil, olive oil, and the like. Organic esters such as ethyl oleate are also suitable non-aqueous carriers. Aqueous carriers include water, ethanol, glycerol, alcoholic/aqueous solutions, emulsions, or suspensions, including saline and buffered media. Water or an aqueous carrier is preferred when the composition is a pharmaceutical composition that is administered intravenously. Saline solutions and aqueous dextrose and glycerol solutions can also be employed as liquid carriers, particularly for injectable solutions. The composition, if desired, can also contain wetting or emulsifying agents, lubricants, glidants, emollients, humectants, thickeners, flavoring agents, preservatives, or pH buffers. pH buffers can be included to control the pH of the composition. In some examples, the buffer is included to maintain the pH of the composition between 5 and 8.5, which can dictate the rate of nitric oxide (NO) release. For example, the buffer can be included to maintain the pH of the composition between 5.2 and 8.3, 5.5 and 8.0, 6.0 and 8.0, 6.8 and 8.0 or between 7.0 and 7.8 (e.g., 7.4). Examples of suitable buffers include phosphate buffers such as phosphate buffered saline (PBS), e.g., 0.01-0.1 M phosphate buffers, acetate buffers, benzoate buffers, citrate buffers, lactate buffers, maleate buffers, and tartrate buffers. Buffered carriers like Hanks's solution, Ringer's solution, dextrose solution, 5% human serum albumin, Ringer's dextrose, dextrose and sodium chloride, lactated Ringer's and fixed oils, polyethylene glycol, polyvinyl pyrrolidone, or lecithin can be used. Monoethanolamine, diethanolamine, tromethamine, and glycine solutions can also be used as suitable buffers. Liposomes and nonaqueous vehicles such as fixed oils may also be used as carriers. The formulation should suit the mode of administration. Additional carriers for use in the compositions are described in the pharmaceutical compositions section herein.

Furthermore, the one or more compounds, compositions, and formulations described herein can be combined with other agents, including treatments for lung, digestive, hepatic, and biliary tract related diseases and disorders. For example, in the case of cystic fibrosis, the compositions and formulations described herein can be combined with mucus thinning drugs (e.g., dornase alfa, N-acetyl cysteine, and hypertonic saline), bronchodilators (e.g., metaproterenol sulfate, pirbuterol acetate, salmeterol, albuterol, and terbutaline sulfate), P2Y2-receptor agonists (e.g., denufosol), and agents that target nonsense mutations (e.g., PTC124). Further examples of additional agents that can be combined with the compounds described herein include additional antibiotics (e.g., aminoglycosides, antipseudomonal penicillins, and cephalosporins), additional antimicrobial drugs (e.g., rifabutin), ethambutol, clarithromycin, clofazimine, aztreonam, steroidal and nonsteroidal anti-inflammatory drugs (e.g., ibuprofen and prednisone), pentoxifylline, dornase alfa, or ursodeoxycholic acid.

The one or more compounds and compositions described herein, with or without additional agents, can be provided in the form of an inhaler or nebulizer for inhalation therapy. As used herein, inhalation therapy refers to the delivery of a therapeutic agent, such as the compounds and compositions described herein, in an aerosol form to the respiratory tract (i.e., pulmonary delivery). As used herein, the term aerosol refers to very fine liquid or solid particles carried by a propellant gas under pressure to a site of therapeutic application. When a pharmaceutical aerosol is employed, the aerosol contains the one or more compounds and compositions described herein, which can be dissolved, suspended, or emulsified in a mixture of a fluid carrier and a propellant. The aerosol can be in the form of a solution, suspension, emulsion, powder, or semi-solid preparation. In the case of a powder, no propellant gas is required when the device is a breath activated dry powder inhaler. Aerosols employed are intended for administration as fine, solid particles or as liquid mists via the respiratory tract of a patient.

The propellant of an aerosol package containing the one or more compositions described herein can be capable of developing pressure within the container to expel the compound when a valve on the aerosol package is opened. Various types of propellants can be utilized, such as fluorinated hydrocarbons (e.g., trichloromonofluoromethane, dichlorodifluoromethane, and dichlorotetrafluoroethane) and compressed gases (e.g., nitrogen, carbon dioxide, nitrous oxide, or Freon). The vapor pressure of the aerosol package can be determined by the propellant or propellants that are employed. By varying the proportion of each component propellant, any desired vapor pressure can be obtained within the limits of the vapor pressure of the individual propellants.

As described above, the one or more compositions described herein can be provided with a nebulizer, which is an instrument that generates very fine liquid particles of substantially uniform size in a gas. The liquid containing the one or more compounds and/or compositions described herein can be dispersed as droplets about 5 mm or less in diameter in the form of a mist. The small droplets can be carried by a current of air or oxygen through an outlet tube of the nebulizer. The resulting mist can penetrate into the respiratory tract of the patient.

Additional inhalants useful for delivery of the compositions described herein include intra-oral sprays, mists, metered dose inhalers, and dry powder generators (See Gonda, J. Pharm. Sci. 89:940-945, 2000, which is incorporated herein by reference in its entirety, at least, for inhalation delivery methods taught therein). For example, a powder composition containing the one or more compounds as described herein, with or without a lubricant, carrier, or propellant, can be administered to a patient. The delivery of the one or more compounds in powder form can be carried out with a conventional device for administering a powder pharmaceutical composition by inhalation.

Depending on the intended mode or modes of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

The preparation of pharmaceutically acceptable carriers and formulations containing these materials is described in, e.g., Remington: The Science and Practice of Pharmacy, Adeboye Adejare ed., 23rd Ed., Academic Press (2021). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds and compositions described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds and compositions described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds and compositions described herein or derivatives thereof include ointments, powders, sprays, inhalants, gels, pastes, creams, and lotions. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

As noted above, the compositions can include one or more of the compounds described herein or pharmaceutically acceptable salts thereof. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.01 to about 200 mg/kg of body weight of active compound per day, which may be administered in a single dose or in the form of individual divided doses, such as from 1 to 4 times per day. Alternatively, the dosage amount can be from about 0.05 to about 190 mg/kg of body weight of active compound per day, about 0.1 to about 180 mg/kg of body weight of active compound per day, about 0.25 to about 175 mg/kg of body weight of active compound per day, about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### IV. Methods of Use

Methods of treating a microbial infection in a subject are provided herein. The methods include administering to the subject compositions of a multi-component pharmaceutical composition or kit as described herein. The method can comprise administering a first component comprising a nitric oxide (NO) releasing compound as described herein (and optionally having antimicrobial characteristics, including antiviral, antibacterial, and antifungal characteristics, anti-inflammatory properties, and/or other beneficial therapeutic properties) and administering to the subject a second component comprising a second therapeutic agent, wherein the second therapeutic agent comprises an antibiotic, an antifungal agent, or a combination thereof.

In some cases of these methods, a concentration of the second therapeutic agent in the second component administered to the subject can be lower than the concentration of the second therapeutic agent needed alone to exhibit an antimicrobial effect against a microbe in the subject. In some cases, the concentration of the second therapeutic agent administered to the subject is at least 10% lower, at least 20% lower, or at least 30% lower than the concentration of the second therapeutic agent needed alone to exhibit an antimicrobial effect against a microbe in the subject. Even still, the compositions described herein are as effective as or more effective than the second therapeutic agent administered alone. In some cases, the NO releasing compound sensitizes or re-sensitizes a microbe to the second therapeutic agent, such that the second therapeutic agent exhibits renewed or greater antimicrobial effects.

The NO releasing compound and the second therapeutic agent are administered to the subject by any suitable method, including (as further described herein) orally, parenterally, intravenously, via inhalation, intraperitoneally, intracranially, intraspinally, intrathecally, intraventricularly, intramuscularly, subcutaneously, sublingually, buccally, intracavitarly or transdermally. The NO releasing compound and the second therapeutic agent can be administered using the same mode of administration or via different modes of administration. In some instances, the NO releasing compound and the second therapeutic agent are administered simultaneously, whereas in other instances, the NO releasing compound and the second therapeutic agent are administered sequentially. Optionally, the NO releasing compound is administered prior to administering the second therapeutic agent. In some cases, the NO releasing compound is administered 24 hours or less, 12 hours or less, 11 hours or less, 10 hours or less, 9 hours or less, 8 hours or less, 7 hours or less, 6 hours or less, 5 hours or less, 4 hours or less, 3 hours or less, 2 hours or less, or 1 hour or less prior to administering the second therapeutic agent to the subject.

Optionally, the microbial infection treated by the compositions described herein can be a bacterial infection. The bacterial infection can be caused by Gram-positive bacteria, Gram-negative bacteria, or atypical bacteria. In some instances, the bacterial infection is caused by Gram-positive bacteria species, such as an Actinomyces species, a Bacillus species, a Clostridium species, a Corynebacterium species, an Enterococcus species, a Leuconostoc species, a Micrococcus species, a Nocardia species, a Propionibacterium species, a Staphylococcus species, or a Streptococcus species.

Optionally, the bacterial infection can be caused by Gram-negative bacteria species, such as an Acinetobacter species, an Aeromonas species, an Alcaligenes/Achromobacter species, a Bacteroides species, a Bartonella species, a Bordetella species, a Borrelia species, a Brevundimonas species, a Brucella species, a Burkholderia species, a Campylobacter species, a Citrobacter species, a Coxiella species, an Ehrlichia species, an Enterobacter species, an Escherichia species, a Francisella species, a Haemophilus species, a Helicobacter species, a Klebsiella species, a Leclercia species, a Legionella species, a Leptospira species, a Listeria species, a Moraxella species, a Morganella species, a Neisseria species, an Orientia species, a Pantoea species, a Paracoccus species, a Prevotella species, a Proteus species, a Providencia species, a Pseudomonas species (e.g., Pseudomonas aeruginosa), a Ralstonia species, a Rickettsia species, a Roseomonas species, Salmonella species, a Serratia species, a Shigella species, a Sphingomonas species, a Stenotrophomonas species, a Treponema species, a Ureaplasma species, a Vibrio species, or a Yersinia species.

Optionally, the bacterial infection can be caused by an atypical bacteria species, such as a Mycobacteria species, a Chlamydia/Chlamidophila species, or a Mycoplasma species. In some cases, the bacterial infection can be caused by or can develop into antibiotic-resistant bacteria, such as antibiotic-resistant Burkholderia cepacia, carbapenem-resistant Enterobacteriaceae (CRE) gut bacteria, drug-resistant Campylobacter, drug-resistant non-typhoidal Salmonella, drug-resistant Shigella, multi-drug-resistant Acinetobacter, multi-drug-resistant Escherichia coli, multi-drug-resistant Klebsiellapneumoniae, multi-drug-resistant Neisseria gonorrhoeae, multidrug-resistant Pseudomonas aeruginosa, antibiotic-resistant Clostridium difficile, drug-resistant Streptococcus pneumoniae, clindamycin-resistant Group B Streptococcus, erythromycin-resistant Group A Streptococcus, methicillin-resistant Staphylococcus aureus (MRSA), vancomycin-resistant Staphylococcus aureus (VRSA), and vancomycin-resistant Enterococcus (VRE).

Also provided herein are methods of preventing, reducing, or eliminating biofilm formation caused by bacteria. The methods include contacting bacteria with an effective amount of composition(s) of a multi-component pharmaceutical composition or kit as described herein. The effective amount of the composition(s) can be the amount that prevents, reduces, and/or eliminates bacterial biofilm formation, and includes a concentration of the second therapeutic agent administered to the subject is lower than the concentration of the second therapeutic agent needed alone to exhibit an antimicrobial effect against the bacteria.

The compositions described herein are useful for preventing, reducing, or eliminating biofilm formation caused by bacteria in humans, e.g., pediatric and geriatric populations, in animals, e.g., veterinary applications, and on surfaces, e.g., medical device surfaces.

The methods described herein can further include selecting a subject infected with or at risk of being infected with a bacterium. Optionally, the methods can further include selecting a subject infected with or at risk of being infected with a bacterium that is capable of developing resistance to an antibiotic. Subjects at risk of being infected with a bacterium as described above include young children, the elderly, immuno-compromised subjects, hospitalized subjects, subjects living in institutions (e.g., nursing homes), subjects having an invasive medical device (e.g., a urinary catheter), subjects having open wounds, and subjects that have come into contact with others infected with the bacteria.

The methods of treatment or prevention described herein can further include treatment with one or more additional agents (e.g., a second biofilm inhibiting agent). As noted above, the one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different modes. When treating with one or more additional agents, the compounds and compositions or pharmaceutically acceptable salts thereof as described herein can be combined into a pharmaceutical composition that includes the one or more additional agents.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of bacterial biofilm formation), during early onset (e.g., upon initial signs and symptoms of bacterial biofilm formation), or after an established formation of a bacterial biofilm. Prophylactic administration can occur for several hours to years prior to the manifestation of symptoms of an infection. Prophylactic administration can be used, for example, in the preventative treatment of subjects or surfaces exposed to Pseudomonas aeruginosa. Therapeutic treatment involves contacting the bacteria with a therapeutically effective amount of the compositions as described herein after a bacterial biofilm formation is observed.

Optionally, the contacting can be performed under aerobic conditions (also referred to as in an aerobic environment). The term aerobic conditions, as used herein, refers to conditions characterized by the presence of free oxygen (O₂). Optionally, the contacting can be performed under anaerobic conditions (also referred to as in an anaerobic environment). The term anaerobic conditions, as used herein, refers to conditions lacking free oxygen (O₂). Optionally, the contacting can be performed under microaerobic conditions (also referred to as in a microaerobic environment). The term microaerobic conditions, as used herein, refers to conditions having low levels of free oxygen (O₂), meaning below normal atmospheric oxygen levels and between aerobic and anaerobic conditions.

Also provided herein are methods of treating a surface to prevent, reduce, or eliminate biofilm formation caused by bacteria. The methods of treating a surface to prevent, reduce, or eliminate bacterial biofilm formation include contacting the surface with an effective amount of composition(s) of the multi-component pharmaceutical composition or kit as described herein. The effective amount of the composition can be the amount that prevents, reduces, and/or eliminates bacterial biofilm formation on a surface, and includes a concentration of the second therapeutic agent in the second component that is lower than the concentration of the second therapeutic agent needed alone to exhibit an antimicrobial effect against the bacteria. Optionally, the surface is a human body surface, such as a mucosal surface. Optionally, the mucosal surface is a mucosal surface of the lungs or upper airways.

### V. Kits for Treating Microbial Infections and Preventing, Reducing, or Eliminating Biofilm Formation

Also provided herein are kits for treating a microbial infection in a subject; for preventing, reducing, or eliminating biofilm formation caused by bacteria; and also kits for treating or pretreating a surface to prevent, reduce, or eliminate biofilm formation caused by bacteria. A kit can include any of the compositions described herein. For example, a kit can include an NO releasing agent (e.g., a compound of Formula I) and a second therapeutic agent. Optionally, the kit can further include a carrier (e.g., a pharmaceutically acceptable carrier).

A kit can include a means for delivery by inhalation (e.g., an inhaler or a nebulizer) or an oral formulation of any of the compositions described herein. A kit can additionally include directions for use of the kit (e.g., instructions for treating a subject or contacting a surface), one or more containers (for the compound(s), composition(s), or second biofilm inhibiting agent(s), a means for administering the compounds or compositions, and/or a carrier.

Optionally, the multi-component kit can include one or more containers. A kit can include a first container including the first component comprising a nitric oxide releasing compound. Optionally, the kit can include a second container including a second therapeutic agent. Optionally, the kit can include a third container for combining the components of the first and second containers for optional use in cases where the first and second components are administered as a single, combined composition.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs of the disease in a subject as compared to a control. As used herein, control refers to the untreated condition. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

### Example 1: Efficacy of NO Releasing Compounds in Combination with Other Antibiotics for Treating P. aeruginosa Lung Infections

Patients suffering from certain infections, such as chronic lung infections, are often prescribed multiple antibiotics simultaneously. Therefore, it is important to evaluate whether NO releasing agents alter the efficacy of commonly used antibiotics, and whether commonly used antibiotics affect the efficacy of NO releasing agents. For purposes of this study, MD3 is used as the representative NO releasing agent.

To quantify the combined effects of MD3 and a secondary antibiotic, a checkerboard assay was used, which is a two-dimensional, two-agent serial dilution that quantifies the effect of each drug alone and in combination. In patients with underlying pulmonary disease such as bronchiectasis, significant mucus in the lung results in bacterial infections that are primarily microaerobic (little oxygen) or anaerobic (no oxygen). Therefore, combinations of MD3 and six secondary antibiotics were evaluated under both aerobic and anaerobic conditions for a representative organism, Pseudomonas aeruginosa.

The antibiotics tested in this study were selected to represent diverse mechanisms of action for antibiotics used to treat pulmonary P. aeruginosa infections, shown in Table 1.

TABLE 1
Antibiotics used in this study

| Antibiotic | | | Mechanism |
|---|---|---|---|
| Name | Abbreviation | Class | of action |
| Amikacin | AMK | Aminoglycoside | Inhibits protein |
| | | | synthesis |
| | | | (30s ribosome) |
| Aztreonam | ATM | Monobactam | Inhibits cell wall |
| | | | synthesis |
| Cetftazidime | CAZ | Cephalosporin | Destroys bacterial |
| | | (3^{rd} generation) | cell wall |
| Ciprofloxacin | CIP | Quinolone | Inhibits DNA |
| | | | replication |
| Clarithromycin | CLR | Macrolide | Inhibits protein |
| | | | synthesis |
| | | | (50s ribosome) |
| Colistin | CST | Polymyxin | Disrupts the |
| | | | bacterial cell |
| | | | membrane |
| Gallium citrate | GaCi | None | Iron mimetic |
| Gentamicin | GEN | Aminoglycoside | Inhibits protein |
| | | | synthesis |
| | | | (30s ribosome) |
| Meropenem | MER | Carbapenem | Inhibits cell wall |
| | | | synthesis |
| Tobramycin | TOB | Aminoglycoside | Inhibits protein |
| | | | synthesis (30s |
| | | | and 50s ribosome) |

### Methods

### Species and Strains

The strains used in these studies, along with their sources, are shown in Table 2.

TABLE 2
Species and strains used in this study

| Strain | | | | |
|---|---|---|---|---|
| ID | Species | Strain | Notes | Source |
| N0049 | Pseudomonas | PAK | Clinical isolate | Dr. |
| | aeruginosa | | | Schoenfisch |
| | | | | (UNC) |
| N0040 | Staphylococcus | HI4891 | Clinical isolate; | BcRLR |
| | aureus | | MDR; MRSA | |
| N0010 | Mycobacterium | Isolate #21 | Clinical isolate; | Dr. |
| | abscessus | | smooth | Schoenfisch |
| | | | morphology | (UNC) |
| N0046 | Mycobacterium | 103 | Clinical isolate; | Dr. Ordway |
| | abscessus subsp. | (NR-44261) | mixed | (CSU) |
| | bolletii | | morphology | |
| N0124 | Mycobacterium | NTM0789 | Smooth | BcRLR |
| | chimaera | | morphology | |
| N0130 | Mycobacterium | NTM1244 | Smooth | BcRLR |
| | massiliense | | morphology | |

Abbreviations: UNC, University of North Carolina at Chapel Hill; BcRLR = Burkholderia cepacia Research Laboratory and Repository; CSU, Colorado State University; MDR, multidrug resistant; MRSA, methicillin-resistant S. aureus.

### Checkerboard Assay

The checkerboard method was performed as follows. MD3 and the second antibiotic were tested at four times (4×) their minimum inhibitory concentration (MIC) alone (see Table 3). Stock solutions of MD3 and the secondary antibiotics were prepared at 4× the final concentration (i.e., 16× the MIC; called the 4× stocks).

MD3 and secondary antibiotics were tested at 4× their minimum inhibitory concentration (MIC) alone. For example, the MIC_{MD3} against P. aeruginosa strain PAK grown aerobically is 31.25 μg/ml, and the MIC_{TOB} is 3.125 μg/ml; thus, the highest concentrations tested in the checkerboard assay were 125 g/ml MD3 and 12.5 μg/ml TOB. MD3 and antibiotics were tested across a 7-point range of 2-fold concentration, as detailed in Table 3.

TABLE 3
Concentration tested in the checkerboard
assay plates, shown in μg/ml.

| | Strain | | Concentrations |
|---|---|---|---|
| Species | ID | Agent | tested (μg/ml) |
| P. aeruginosa | N0049 | MD3 | 3.91-250 |
| (aerobic) | | TOB | 0.049-3.125 |
| | | ATM | 0.39-25 |
| | | CIP | 0.012-0.78 |
| | | CST | 0.02-6.25 |
| | | CAZ | 0.39-25 |
| | | MER | 0.049-3.125 |
| P. aeruginosa | N0049 | MD3 | 2.0-125 |
| (anaerobic) | | TOB | 0.2-12.5 |
| | | ATM | 0.39-25 |
| | | CIP | 0.012-0.78 |
| | | CST | 0.02-6.25 |
| | | CAZ | 0.2-12.5 |
| | | MER | 0.0061-0.391 |
| S. aureus | N0040 | MD3 | 7.8-500 |
| | | TOB | 0.012-0.78 |
| M. abscessus | N0010 | MD3 | 31.25-2000 |
| | | AMK | 0.25-16 |
| | N0046 | MD3 | 31.25-2000 |
| | | AMK | 0.0020-16 |
| M. chimaera | N0124 | MD3 | 31.25-2000 |
| | | AMK | 0.001-0.064 |
| | | CLR | 0.0003-0.016 |
| M. massiliense | N0130 | MD3 | 31.25-2000 |
| | | AMK | 0.001-0.064 |
| | | CLR | 0.0001-0.0005 |

Abbreviations: TOB, tobramycin; ATM, aztreonam; CIP, ciprofloxacin; CST, colistin; CAZ, ceftazidime; MER, meropenem; AMK, amikacin; CLR, clarithromycin.

Bacterial cultures were prepared from frozen glycerol stocks stored at −80° C. by streaking on agar plates (see Table 4). Several well-isolated colonies were suspended in IX PBS, then diluted to 1×10⁶ CFU/ml in liquid media. The cultures were diluted to 5×10⁵ CFU/ml, when added to the antibiotic combinations in the checkerboard assay plate. Because colonies from strains N0010, N0046, and N0124 are difficult to suspend homogenously, they were first sub-cultured in 7H9 liquid media containing Middlebrook ADC supplement and 0.05% Tween-80, then incubated at 37° C. shaking for 3 days. This subculture was used to prepare the 1×10⁶ CFU/ml culture. Note: Tween-80 promotes homogenous growth; however, it is not included in the checkerboard assay medium because it has been shown to affect Mycobacterium susceptibility to antimicrobials (Van Boxtel, 1990).

To prepare the checkerboard assay plates, phosphate buffered saline (PBS) was added to wells BI through H8 of a 96-well plate. Next, 200 μl of the 4× second antibiotic stock was added to the empty wells in A1 through A8 of a 96-well plate, then serially diluted 1:1 down the plate using a multichannel pipette, stopping at row G. The final 100 μl from row G was discarded. Row H did not contain secondary antibiotic—it measures the MIC of MD3 alone. At this step, all wells contain 4× the target concentration of the second antibiotic. Next, 100 μl of the 4× MD3 stock solution was added to wells A1 through H1 and serially diluted 1:1 across the plate to column 7 using a multichannel pipette. The final 100 μl in row 7 was discarded. Column 8 did not contain any MD3—it measures the MIC of the secondary antibiotic alone. After this step, all wells contain 2× the target concentrations for both MD3 and the second antibiotic. The wells in column 9 contain media alone, serving as media controls. The wells in column 10 contain media and bacteria, serving as growth controls. Finally, 100 μl of 5×10⁵ CFU/ml bacterial culture was added to each well. The checkerboard assay plates were incubated at 37° C. until growth was observed in the growth control wells (see Table 4).

For experiments in which P. aeruginosa was grown under anaerobic conditions, 90 mM KNO₃ was added to the CAMHB media, and the checkerboard assay plates were incubated in an anaerobic box containing anaerobic sachets. Anaerobiosis was confirmed via co-incubated anaerobic test strips.

TABLE 4
Checkerboard assay conditions used.

| | Agar | Agar | Checkerboard | Checkerboard |
|---|---|---|---|---|
| Species | plates | incubation | media | incubation |
| Pseudomonas | MHA | 37° C., | CAMHB | 37° C., |
| aeruginosa | | 1 day | | 1 day |
| Staphylococcus | MHA | 37° C., | CAMHB | 37° C., |
| aureus | | 1 day | | 1 day |
| Mycobacterium | BHI | 37° C., | CAMHB | 37° C., |
| abscessus | agar | 4 days | | 5-7 days |
| Mycobacterium | BHI | 37° C., | CAMHB | 37° C., |
| abscessus subsp. | agar | 4 days | | 5-7 days |
| bolletii | | | | |
| Mycobacterium | 7H11 + | 37° C., | 7H9 + ADC | 37° C., |
| chimaera | OADC | 8-10 days | | 10-14 days |
| Mycobacterium | 7H11 + | 37° C., | CAMHB | 37° C., |
| massiliense | OADC | 3-5 days | | 5-7 days |

Abbreviations: MHA, Mueller Hinton agar; CAMHB, cation-adjusted Mueller Hinton broth; BHI, brain heart infusion; OADC; Middlebrook supplement containing oleic acid, bovine albumin, dextrose, and catalase; ADC, Middlebook supplement containing bovine albumin, dextrose, and catalase.

### Interpretation of Checkerboard Assay Results

The effects of the antibiotic combinations were classified according to the fractional inhibitory concentration index (FICI) as described by Chou and Talay (1984) using Equation 1, where MIC_{A} and MIC_{B} are the values determined for agents A and B when tested alone, respectively, and MIC_{AB} and MIC_{BA} are the concentrations of agent A and B, respectively, in the most effective combination in the checkerboard assay.

$$FICI = \frac{{MIC}_{A B}}{{MIC}_{A}} + \frac{{MIC}_{B A}}{{MIC}_{B}} \qquad Eq . 1$$

The FICI was calculated for each well along the growth/no-growth border of the checkerboard assay plate. If the FICI of any well was greater than 4, antagonism was reported; otherwise, the lowest FICI value was reported. FICI values of ≤0.5 are considered synergistic, values of 0.51 to 1 are additive, and values of greater than 1 to 4 are indifferent. The rationale for this method of analysis is that, if any ratio of MD3:antibiotic is synergistic, then that combination has the potential for synergy, even if other ratios are not synergistic. The classifications of synergy and antagonism was only be reported if it was observed in a minimum of two biological replicates. If an experiment was repeated twice and the same classification observed (e.g., synergy observed in both replicates), then the average FICI was reported for that drug combination. If the results for two duplicates are discordant in their classification, a third experiment must be conducted and the average FICI from the triplicate experiments was used for final classification.

### Assay Validity

A checkerboard assay was considered valid if all of the following statements were true:
The starting inoculum was confirmed to be between 1×10⁵-9.0×10⁶ CFU/ml;
The starting inoculum produced pure, single colonies on agar;
Growth was observed in the growth control wells within a species-appropriate amount of time, as described in the ‘bacterial cultures’ section;
At least 7 out of 8 growth control wells were turbid;
At least 7 out of 8 media control wells were clear; and
The MIC of each agent alone was ±2 wells of the pre-established MIC for that agent against that strain.

### Results

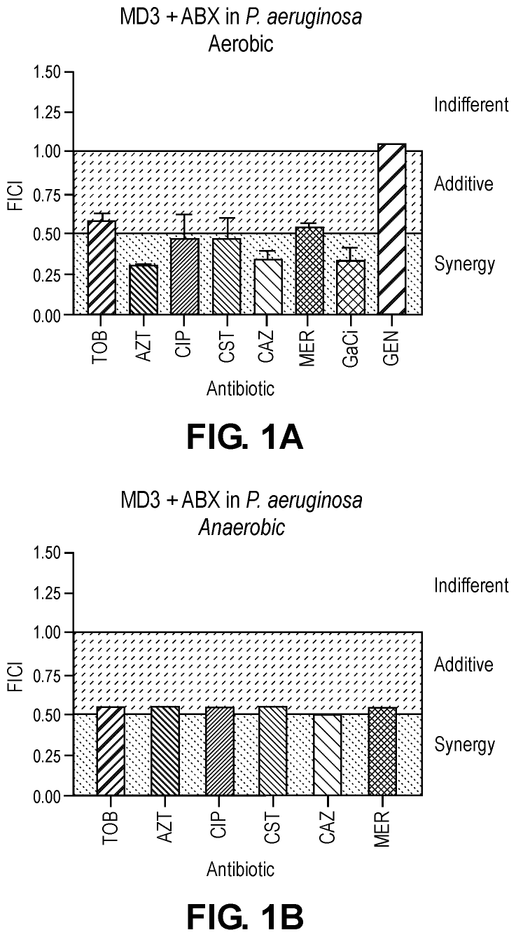

The checkerboard assay results for MD3 combined with secondary antibiotics tested against P. aeruginosa are shown in FIGS. 1A and 1B. Under aerobic conditions, MD3 is synergistic with aztreonam, ciprofloxacin, colistin, ceftazidime, and gallium citrate, additive with tobramycin and meropenem, and indifferent with gentamicin (FIG. 1A). Under anaerobic conditions, MD3 was additive with all six tested antibiotics, tobramycin, aztreonam, ciprofloxacin, colistin, ceftazidime, and meropenem. Importantly, no antagonism was observed for any MD3/antibiotic combination tested under either aerobic or anaerobic conditions. See Table 5.

TABLE 5
FICI values obtained from checkerboard assays for P. aeruginosa.

| | Antibiotic | Lowest | Lowest | Lowest |
|---|---|---|---|---|
| Growth | combined | FICI, | FICI, | FICI, |
| condition | with MD3 | rep 1 | rep 2 | rep 3 |
| Aerobic | ATM | 0.31 | 0.31 | NT |
| | CAZ | 0.38 | 0.31 | NT |
| | CIP | 0.56 | 0.31 | 0.56 |
| | CST | 0.56 | 0.38 | NT |
| | GaCi | 0.38 | 0.38 | 0.25 |
| | GEN | 1.06 | NT | NT |
| | MER | 0.53 | 0.56 | NT |
| | TOB | 0.56 | 0.63 | 0.56 |
| Anaerobic | ATM | 0.56 | NT | NT |
| | CAZ | 0.50 | NT | NT |
| | CIP | 0.56 | NT | NT |
| | CST | 0.56 | NT | NT |
| | MER | 0.56 | NT | NT |
| | TOB | 0.56 | NT | NT |

Abbreviations: ATM, aztreonam; CAZ, ceftazidime; CIP, ciprofloxacin; CST, colistin sulfate; GaCi, gallium citrate; GEN, gentamicin; MER, meropenem; TOB, tobramycin. NT, not tested.

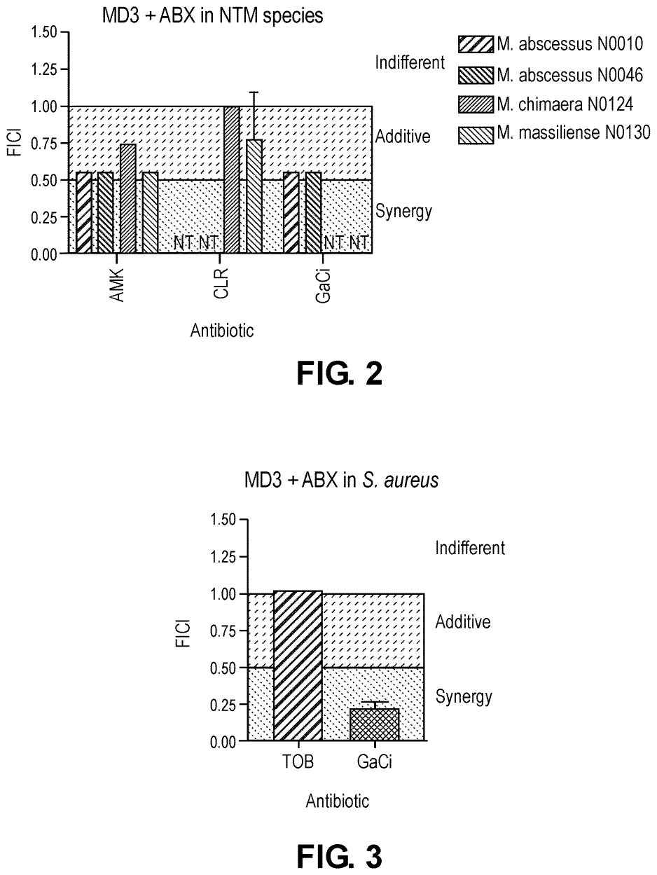

The checkerboard assay results for MD3 combined with secondary antibiotics tested against NTM species are shown in FIG. 2. MD3 was additive with amikacin when used on M. abscessus strains N0010 and N0046, M. chimaera, and M. massiliense. MD3 and clarithromycin was additive when used on M. chimaera, and M. massiliense. MD3 and gallium citrate was additive when used on M. abscessus strains N0010 and N0046. Importantly, no antagonism was observed for any tested combination. See Table 6.

TABLE 6
FICI values obtained from checkerboard assays for NTM.
These were all performed under aerobic conditions.

| | | Antibiotic | Lowest | Lowest |
|---|---|---|---|---|
| | | combined | FICI, | FICI, |
| Species | Strain | with MD3 | rep 1 | rep 2 |
| M. abscessus | N0010 | AMK | 0.56 | NT |
| | N0046 | AMK | 0.56 | NT |
| | N0010 | GaCi | 0.56 | NT |
| | N0046 | GaCi | 0.56 | NT |
| M. chimaera | N0124 | AMK | 0.75 | NT |
| | N0124 | CLR | 1.00 | NT |
| M. massiliense | N0130 | AMK | 0.56 | 0.56 |
| | N0130 | CLR | 0.56 | 1.00 |

Abbreviations: AMK, amikacin; GaCi, gallium citrate; CLR, clarithromycin. NT, not tested.

Finally, the checkerboard assay results for MD3 combined with secondary antibiotics tested against S. aureus are shown in FIG. 3. MD3 was indifferent with tobramycin and synergistic with gallium citrate when used on S. aureus strain N0040. Again, no antagonism was observed for any tested combination. See Table 7.

TABLE 7
FICI values obtained from checkerboard assays for S. aureus.
These were all performed under aerobic conditions.

| | | Antibiotic | Lowest | Lowest |
|---|---|---|---|---|
| | | combined | FICI, | FICI, |
| Species | Strain | with MD3 | rep 1 | rep 2 |
| S. aureus | N0040 | GaCi | 0.25 | 0.19 |
| | | TOB | 1.03 | NT |

Abbreviations: GaCi, gallium citrate; TOB, tobramycin. NT, not tested.

In summary, the efficacy of MD3 in combination with other antibiotics commonly used to treat pulmonary infections was studied to evaluate any potential drug-drug interactions. Studies were conducted primarily in P. aeruginosa, the most common and most detrimental pathogens for patients with underlying lung disease such as bronchiectasis of cystic fibrosis. In studies with P. aeruginosa, antibiotics spanning six classes of antibiotics were evaluated under both aerobic and anaerobic conditions. MD3/antibiotic combinations show no antagonism, suggesting MD3 would be a good candidate for combination therapy. In fact, a synergistic effect was observed when MD3 was combined with aztreonam, ciprofloxacin, colistin, ceftazidime, and gallium citrate, and an additive effect was observed when MD3 was combined with tobramycin and meropenem. These results support the potential combination of MD3 with one of these traditional antibiotics for the management of P. aeruginosa infection.

Additional studies were undertaken to evaluate the combination of MD3 and antibiotics commonly used to treat NTM infections, which are typically treated with a minimum of three antibiotics at a time for at least six months. The results of these studies indicate that MD3 is additive with amikacin and clarithromycin, two of the most commonly prescribed antibiotics for NTM lung infections. These results support the potential combination of MD3 and amikacin or MD3 and clarithromycin for the treatment of NTM infection.

An additional study was conducted to evaluate drug-drug interactions of a Gram-positive organism, S. aureus. In this species, MD3 combined with tobramycin was indifferent, suggesting that combination of MD3 with tobramycin should not diminish the efficacy of either drug.

### Example 2: Efficacy of NO Releasing Compounds in Combination with Other Antifungals Against Aspergillus fumigatus and Candida auris

As detailed above in Example 1 with respect to antibiotics, it is important to evaluate whether NO releasing agents alter the efficacy of commonly used antifungals, and whether commonly used antifungals affect the efficacy of NO releasing agents. For purposes of this study, MD3 is used as the representative NO releasing agent.

To quantify the combined effects of MD3 and a secondary antifungal, a checkerboard assay was used as described above in Example 1. The antibiotics tested in this study included voriconazole, a triazole antifungal that inhibits the growth of the fungi causing the infection; itraconazole, an azole antifungal that inhibits the growth of fungi; and amphotericin B, a polyene antifungal that binds to ergosterol in fungal cell membranes, developing holes in the membrane and allowing cell components to leak out, causing cell death.

The fungal species used in this study included Aspergillus fumigatus (strain N0219) and Candida auris (Strain N0220). The checkerboard method was performed as described above in Example 1. The effects of the antifungal combinations were classified according to the fractional inhibitory concentration index (FICI) as described above in Example 1 at time points of 24 hours and 48 hours. The checkerboard assay results for MD3 combined with secondary antifungals are shown in Table 8.

TABLE 8
FICI values obtained from checkerboard assays for fungal species

| | | | Lowest | Interaction | Lowest | Interaction |
|---|---|---|---|---|---|---|
| | | Antifungal | FICI (at | (at 24 | FICI (at 48 | (at 48 |
| Species | Strain | Tested | 24 hours) | hours) | hours) | hours) |
| Aspergillus | N0219 | Voriconazole | 0.625 | Additive | 0.313 | Additive |
| fumigatus | | | | | | |
| Aspergillus | N0219 | Itraconazole | 0.625 | Additive | 0.5625 | Additive |
| fumigatus | | | | | | |
| Candida auris | N0220 | Voriconazole | 0.375 | Additive | 0.5625 | Additive |
| Candida auris | N0220 | Amphotericin | 0.625 | Additive | 0.5625 | Additive |
| | | B | | | | |

In summary, MD3 is at least additive with voriconazole for Aspergillus fumigatus and Candida auris. In addition, MD3 is at least additive with itraconazole for Aspergillus fumigatus and Candida auris. The combinations of MD3 and the tested antifungals both compounds worked equally well alone and in combination, so the combination is better than a single compound. Importantly, no antagonism was observed for any of the MD3/antifungal combinations tested. See Table 8.

### Example 3: Inhibition of P. aeruginosa Biofilm Formation

A 96-well plate was prepared containing serial dilutions of MD3 or media only (untreated control). The test concentrations of MD3 included 0.00391 mg/mL, 0.00781 mg/mL, 0.0156 mg/mL, 0.3125 mg/mL, and 0.0625 mg/mL. P. aeruginosa, at a concentration of 10⁶ colony forming units (CFU)/mL, was added to the wells. A peg lid was inserted into the plate and biofilms were allowed to grow on the pegs for 18 to 24 hours. The pegs were then rinsed with phosphate-buffered saline (PBS) to remove planktonic bacteria. The biofilms were disrupted and plated to enumerate biofilm-associated colony forming units.

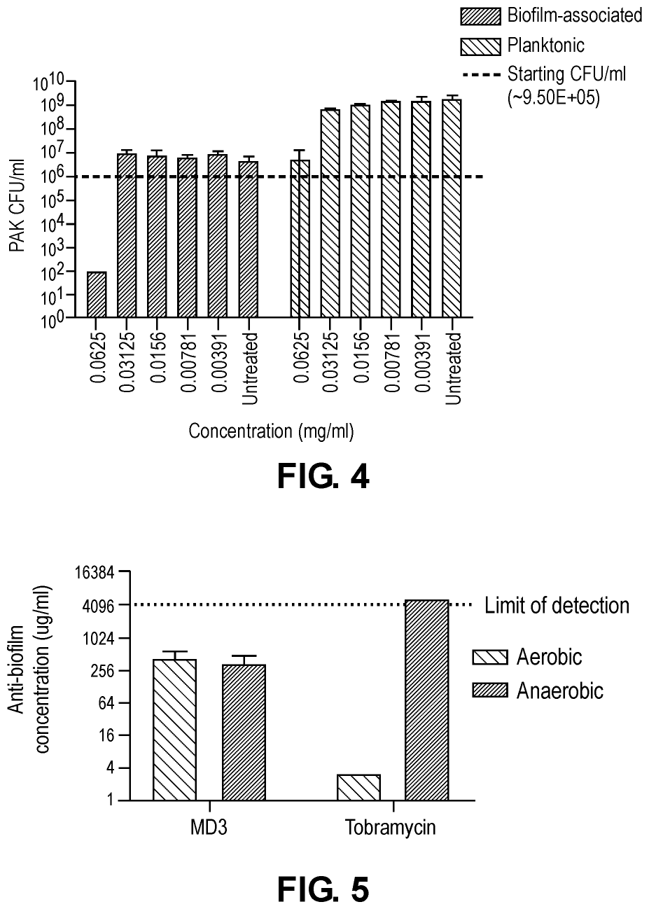

FIG. 4 shows the number of remaining biofilm-associated and planktonic P. aeruginosa bacteria per mL of sample after treatment with varying concentrations of MD3. Biofilm-associated bacteria measurements are shown in the left group of bars (bars 1-6, starting from the left) and planktonic bacteria measurements are shown in the right group of bars (bars 7-12, starting from the left). The starting CFU/mL, prior to treatment with MD3, was 9.50E+05 and is indicated by the dotted line. As shown in FIG. 4, MD3 effectively prevented P. aeruginosa from forming biofilms on pegs at a concentration of 0.0625 mg/mL. Surprisingly, the P. aeruginosa biofilm-prevention concentration of MD3 is approximately two-folds less than the minimum inhibitory concentration (MIC) for MD3 against planktonic P. aeruginosa, which is 0.125 mg/mL. See Table 9.

TABLE 9

| | | Biofilm-Prevention |
|---|---|---|
| Compound | MIC | Concentration |
| MD3 | 0.125 mg/mL | 0.0625 mg/mL |

### Example 4: Eradication of P. aeruginosa Biofilms

The minimum biofilm eradication concentration (MBEC) of MD3 was determined for P. aeruginosa biofilms grown under aerobic and anaerobic conditions. The MBEC is defined as a 3-log reduction in biofilm-associated CFUs. Biofilms were generated using the MBEC Assay™ growth device (Innovotech; Edmonton, Alberta, Canada) in cation-adjusted Mueller-Hinton broth (CAMHB) at 37° C. for 24 hours and treated with MD3 or tobramycin for an additional 18-24 hours. Remaining biofilms were disrupted by sonication and plated to determine the surviving CFU/mL following a single treatment.

As shown in FIG. 5, MD3 eradicated P. aeruginosa biofilms at similar concentrations under both aerobic and anaerobic conditions. Tobramycin, used as a comparative example, was only effective against aerobic biofilms. MD3's potent, broad-spectrum antibacterial activity shows that the compound is effective for treating P. aeruginosa infections.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A pharmaceutical composition, comprising:
a first component comprising a nitric oxide (NO) releasing compound; and
a second component comprising a second therapeutic agent, wherein the second therapeutic agent comprises an antibiotic, an antifungal agent, or a combination thereof,
wherein a ratio of the first component to the second component provides an in vitro fractional inhibitory concentration index (FICI) of a combination of the first component and the second component of 1.0 or lower.

2. The pharmaceutical composition of claim 1, wherein a concentration of the second therapeutic agent in the second component is lower than the concentration of the second therapeutic agent needed alone to exhibit an antimicrobial effect against a microbe.

3. The pharmaceutical composition of claim 2, wherein the concentration of the second therapeutic agent in the second component is at least 10% lower than the concentration of the second therapeutic agent needed alone to exhibit an antimicrobial effect against a microbe.

4. The pharmaceutical composition of claim 2, wherein the concentration of the second therapeutic agent in the second component is at least 20% lower than the concentration of the second therapeutic agent needed alone to exhibit an antimicrobial effect against a microbe.

5. The pharmaceutical composition of claim 2, wherein the concentration of the second therapeutic agent in the second component is at least 30% lower than the concentration of the second therapeutic agent needed alone to exhibit an antimicrobial effect against a microbe.

6. The pharmaceutical composition of claim 1, wherein the NO releasing compound in the first component is present in an effective amount to sensitize or re-sensitize a microbe to the second therapeutic agent in the second component.

7. The pharmaceutical composition of claim 1, wherein the nitric oxide (NO) releasing compound comprises a compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

8. The pharmaceutical composition of claim 7, wherein the compound has the following structure: 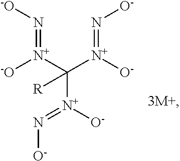
wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M₊ is a pharmaceutically-acceptable cation, wherein a ratio of the compound to the cation is such that the overall net charge of the compound is neutral.

9. The pharmaceutical composition of claim 8, wherein the cation is selected from the group consisting of sodium, potassium, lithium, calcium, magnesium, ammonium, and substituted ammonium.

10. The pharmaceutical composition of claim 8, wherein the compound has the following structure:

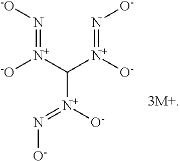

11. The pharmaceutical composition of claim 8, wherein the compound has the following structure:

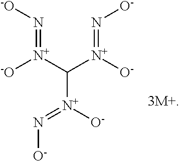

12. The pharmaceutical composition of claim 1, wherein the second therapeutic agent in the second component comprises an antibiotic.

13. The pharmaceutical composition of claim 12, wherein the antibiotic is selected from the group consisting of an aminoglycoside, a monobactam, a cephalosporin, a quinolone, a macrolide, a polymyxin, and a carbapenem.

14. The pharmaceutical composition of claim 1, wherein the second therapeutic agent in the second component comprises an antifungal agent.

15. The pharmaceutical composition of claim 14, wherein the antifungal agent is selected from the group consisting of a polyene, an azole, an allylamine, and an echinocandin.

16. The pharmaceutical composition of claim 1, wherein the in vitro FICI is 0.5 or lower.

17. The pharmaceutical composition of claim 1, wherein the in vitro FICI is 0.3 or lower.

18. The pharmaceutical composition of claim 1, wherein a molar equivalents concentration ratio of the NO releasing compound to the second therapeutic agent is from 0.1:1 to 10:1.

19. The pharmaceutical composition of claim 18, wherein the molar equivalents concentration ratio of the NO releasing compound to the second therapeutic agent is from 0.5:1 to 2:1.

20. The pharmaceutical composition of claim 1, further comprising one or more additives.

21. The pharmaceutical composition of claim 20, wherein the one or more additives comprises one or more preservatives, salts, chelators, viscosity modifiers, stabilizers, surfactants, antioxidants, buffering agents, or cosolvents.

22. A multi-component kit, comprising:
a first component comprising a nitric oxide (NO) releasing compound; and
a second component comprising a second therapeutic agent, wherein the second therapeutic agent comprises an antibiotic, an antifungal agent, or a combination thereof,
wherein a ratio of the first component to the second component provides an in vitro fractional inhibitory concentration index (FICI) of a combination of the first component and the second component of 1.0 or lower.

23-45. (canceled)

46. A method of treating a microbial infection in a subject, comprising administering to the subject a multi-component pharmaceutical composition or kit comprising:
a first component comprising a nitric oxide (NO) releasing compound; and
a second component comprising a second therapeutic agent, wherein the second therapeutic agent comprises an antibiotic, an antifungal agent, or a combination thereof,
wherein a ratio of the first component to the second component provides an in vitro fractional inhibitory concentration index (FICI) of a combination of the first component and the second component of 1.0 or lower.

47-88. (canceled)

89. A method of preventing, reducing, or eliminating biofilm formation caused by bacteria, comprising contacting bacteria with a multi-component kit comprising:
a first component comprising a nitric oxide (NO) releasing compound; and
a second component comprising a second therapeutic agent, wherein the second therapeutic agent comprises an antibiotic, an antifungal agent, or a combination thereof,
wherein a ratio of the first component to the second component provides an in vitro fractional inhibitory concentration index (FICI) of a combination of the first component and the second component of 1.0 or lower.

90. A method of treating a surface to prevent, reduce, or eliminate biofilm formation caused by bacteria, comprising contacting a surface with multi-component kit comprising:
a first component comprising a nitric oxide (NO) releasing compound; and
a second component comprising a second therapeutic agent, wherein the second therapeutic agent comprises an antibiotic, an antifungal agent, or a combination thereof,
wherein a ratio of the first component to the second component provides an in vitro fractional inhibitory concentration index (FICI) of a combination of the first component and the second component of 1.0 or lower.
